(19) United States
(12) Patent Application Publication
(10) Pub. No.: US 2026/0166341 A1
(43) Pub. Date: Jun. 18, 2026

(54) ADAPTIVE LINEAR ACCELERATOR (LINAC) ISOCENTER BASED ON INDIVIDUAL TREATMENT PLAN

(71) Applicant: VARIAN MEDICAL SYSTEMS, INC., Palo Alto, CA (US)

(72) Inventor: Joao SILVEIRA, Zürich (CH)

(73) Assignee: VARIAN MEDICAL SYSTEMS, INC., Palo Alto, CA (US)

(21) Appl. No.: 18/983,405

(22) Filed: Dec. 17, 2024

(51) Int. Cl. A61N 5/10; H05H 9/00

(52) U.S. Cl. CPC A61N 5/1049; A61N 5/1045; A61N 5/1081; H05H 9/00; H05H 2277/11

(57) ABSTRACT

A radiation therapy (RT) system has a C-arm gantry that is rotationally movable to position the gantry at a plurality of gantry angles. At each gantry angle, a linear accelerator (LINAC) in the gantry generates a treatment beam having a central beam axis. Each treatment beam can be assigned a weight in accordance with at least one treatment plan. A controller can position a target relative to the treatment beam based at least in part on the weight assigned by the treatment plan. This positioning of the target based in accordance with the treatment plan provides an adaptive optimal isocenter for the RT system.

### CROSS-REFERENCE TO RELATED APPLICATIONS

The present application is related in subject matter to U.S. patent application Ser. No. ______ (Attorney Docket No. 124-0076-US1) entitled “REDUCTION OF A LINEAR ACCELERATOR (LINAC) ISOCENTER SIZE THROUGH ADAPTIVE CONTROL OF A SOURCE POSITION” and U.S. patent application Ser. No. ______ (Attorney Docket No. 124-0077-US1) entitled “REDUCTION OF A LINEAR ACCELERATOR (LINAC) ISOCENTER SIZE THROUGH ADAPTIVE CONTROL OF A POSITION OF A MULTI-LEAF COLLIMATOR (MLC) OR A COUCH”, which are incorporated herein by reference.

### BACKGROUND

Unless otherwise indicated herein, the approaches described in this section are not prior art to the claims in this application and are not admitted to be prior art by inclusion in this section.

Radiation therapy is a localized treatment for a specific anatomical target (a planning target volume, or PTV), such as a cancerous tumor. Ideally, radiation therapy is performed on the planning target volume that spares the surrounding normal tissue from receiving doses above specified tolerances, thereby minimizing risk of damage to healthy tissue. Prior to the delivery of radiation therapy, an imaging system is typically employed to provide a three-dimensional image of the anatomical target and surrounding area. From such imaging, the size and mass of the anatomical target can be estimated, a planning target volume determined, and an appropriate treatment plan generated using a dedicated treatment planning system (TPS). The TPS has photon- and electron-beam models that accurately represent the beams generated by the radiation therapy delivery system.

Currently, the field of radiation oncology is moving to treating smaller planning target volumes, for example via stereotactic radiosurgery (SRS) and stereotactic radiotherapy (SRT). Stereotactic radiosurgery and stereotactic radiation therapy are advanced forms of radiation therapy that involve delivery of a high radiation dose to a small focused region of a patient's anatomy. Because of the high radiation dose and small target volumes associated with these SRS treatments, high geometric accuracy of the delivered treatment is required. This high geometrical accuracy is required for both the predicted dose distribution provided by the beam model in the TPS and the delivered dose provided by the actual treatment delivery system.

However, it can be challenging to deliver a radiation beam or other type of treatment beam to a target with accuracy. For example, it can be challenging for a radiation therapy system having a linear accelerator (LINAC) to precisely direct a treatment beam onto or near a target region of a patient, with locational/geometric accuracy.

### SUMMARY

According to various embodiments, a radiation therapy (RT) system comprises: a gantry having a linear accelerator, wherein the gantry is rotationally movable to position the gantry at a plurality of gantry angles, wherein at any of the gantry angles, the linear accelerator is configured to generate a treatment beam having a central beam axis, and wherein each treatment beam is assigned a respective weight in accordance with at least one treatment plan; a couch that provides a target where the treatment beam is directed; and a controller operatively coupled to the couch and configured to position the target relative to the treatment beam based at least in part on the weight assigned by the at least one treatment plan, wherein the controller is further configured to maintain the target in a static position relative to the treatment beam during treatment.

According to various embodiments, a radiation therapy (RT) system comprises: a gantry having a linear accelerator, wherein the gantry is rotationally movable to position the gantry at a plurality of gantry angles, wherein at any of the gantry angles, the gantry is configured to direct a treatment beam generated by the linear accelerator towards a target, and wherein the treatment beam at each of the plurality of gantry angles is associated with a weighting strategy specified in at least one treatment plan; a source of the treatment beam at the linear accelerator; a collimator at the gantry and having a field center, wherein a central beam axis of the treatment beam is defined by the source and the field center; a couch that provides the target where the treatment beam is directed; and a controller operatively coupled to the couch, wherein the controller is configured to position the couch to align the target with the central beam axis, wherein the alignment of the target with the central beam axis is based at least in part on the weighting strategy, and wherein the controller is further configured to statically maintain the alignment of the target with the central beam axis during treatment.

According to various embodiments, a computer-implemented method to adapt a location and size of an isocenter in a radiation therapy (RT) system comprises:
determining a weight assigned by at least one treatment plan to a central beam axis of a treatment beam that deviates relative to the isocenter, wherein the RT system includes a gantry having a linear accelerator configured to generate the treatment beam, wherein the gantry is rotationally movable to position the gantry at a plurality of gantry angles, and wherein at any particular gantry angle of the gantry angles, the gantry is configured to provide the treatment beam; positioning a target of the treatment beam relative to the central beam axis based at least in part on the weight assigned by the at least one treatment plan; and statically maintaining the positioning of the target relative to the central beam axis during treatment.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of the present disclosure will become more fully apparent from the following description and appended claims, taken in conjunction with the accompanying drawings. These drawings depict only several embodiments in accordance with the disclosure and are, therefore, not to be considered limiting of its scope. The disclosure will be described with additional specificity and detail through use of the accompanying drawings.

FIG. 1 is a perspective view of a radiation therapy system, according to various embodiments.

FIG. 2 schematically illustrates a side view of the radiation therapy system of FIG. 1, according to various embodiments.

FIG. 3 schematically illustrates a collimator assembly of the radiation therapy system of FIG. 1, according to various embodiments.

FIG. 4 schematically illustrates plan views of a couch of the radiation therapy system of FIG. 1 in various treatment positions, according to various embodiments.

FIGS. 5 and 6 are schematic side views of a C-arm gantry of the radiation therapy system of FIG. 1, according to various embodiments.

FIG. 7 is a schematic side view of a C-arm gantry of the radiation therapy system of FIG. 1 in which a source position is adjusted, according to various embodiments.

FIG. 8 is a schematic side view of the C-arm gantry of the radiation therapy system 100 of FIG. 1 in which a position of a field center of a collimation assembly is adjusted, according to various embodiments.

FIG. 9 schematically illustrates an example of an adaptive adjustment of a source position, according to various embodiments.

FIG. 10 is a diagram illustrating relationships between a source position and a position of a field center, according to various embodiments.

FIG. 11 is a perspective view of an example multi-leaf collimator (MLC) having an adjustable field center, according to various embodiments.

FIG. 12 is a schematic side view of the C-arm gantry of the radiation therapy system of FIG. 1 illustrating positioning of a couch relative to a treatment beam, according to various embodiments.

FIG. 13 is another schematic side view of the C-arm gantry of the radiation therapy system of FIG. 1 illustrating positioning of a couch relative to a treatment beam, according to various embodiments.

FIGS. 14, 15, 16, 17, 18, and 19 are schematic side views of the C-arm gantry of the radiation therapy system of FIG. 1 illustrating positioning of a couch relative to treatment beams based on weights, according to various embodiments.

FIG. 20 is a block diagram illustrating example components in the radiation therapy system of FIG. 1 that may be used to implement positional adjustment, according to various embodiments.

FIG. 21 is a block diagram illustrating a computing device configured to perform various embodiments of the present disclosure.

FIG. 22 is a block diagram of an illustrative embodiment of a computer program product for implementing a method, according to one or more embodiments of the present disclosure.

FIG. 23 is a flowchart of an example method to reduce isocenter size in the RT system of FIG. 1 and to operate for the RT system, according to various embodiments.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. The aspects of the disclosure, as generally described herein, and illustrated in the figures, can be arranged, substituted, combined, and designed in a wide variety of different configurations, all of which are explicitly contemplated and make part of this disclosure. Independent of the grammatical term usage, individuals with male, female or other gender identities are included within the term.

As explained above, for radiation treatments that involve a high radiation dose and/or a small target size, high geometric accuracy of the delivered radiation treatment (e.g., a treatment beam) is required. This high geometric accuracy is challenging for a radiation therapy (RT) system that includes a C-arm gantry having a linear accelerator (LINAC), wherein the gantry is configured to rotate at a plurality of gantry angles. Due to gravity, a gantry head of the gantry bends downward. This downward bending causes central beam axes of treatment beams from the gantry head to be misaligned/offset/deviated relative to an isocenter, at all gantry angles, thereby increasing an isocenter size (maximum delivery error). A source of a treatment beam and a field center of a collimator provide two points that define each central beam axis.

According to various embodiments, a position of the source can be adjusted/changed to tune the central beam axis to the isocenter, thereby compensating for the misalignment/offset/deviation from the isocenter and reducing the isocenter size for purposes of improved geometric accuracy for delivering the treatment beam to a target placed at the isocenter.

According to various embodiments, a position of the field center can be adjusted/changed to tune the central beam axis to the isocenter, thereby also compensating for the misalignment/offset/deviation from the isocenter and reducing the isocenter size for purposes of improved geometric accuracy for delivering the treatment beam to a target placed at the isocenter.

According to various embodiments, the isocenter size may also be reduced by placing the target (by changing a position of a couch on which the target is placed) in coincidence with an impingement location where the central beam axis is offset from the ideal isocenter. According to still further embodiments, the positioning of the target is based on weights of the central beam axes as specified in a treatment plan.

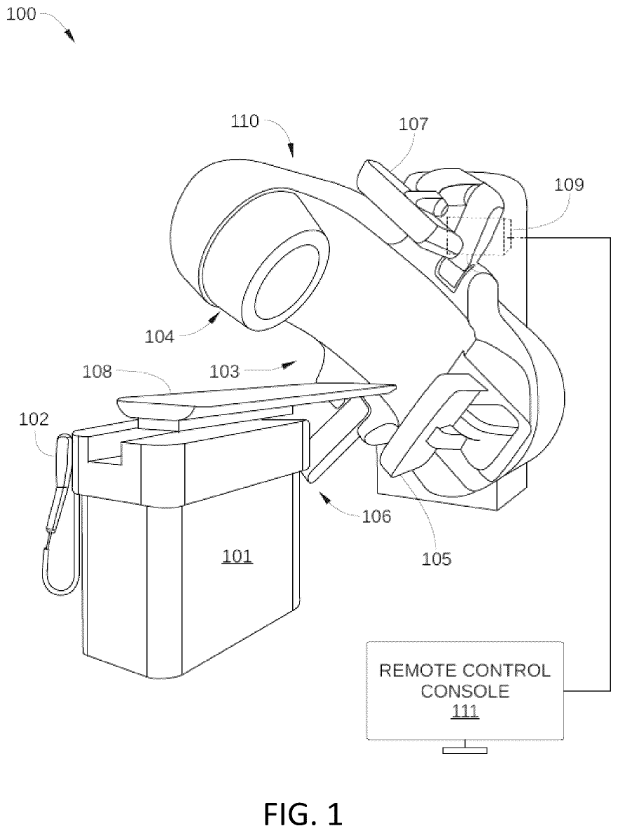

The foregoing and other features of embodiments will be described next below by referring first to FIG. 1 and then to the other figures.

Specifically, FIG. 1 is a perspective view of a radiation therapy (RT) system 100 that can beneficially implement various aspects of the present disclosure. An example of the RT system 100 is a radiation system that may be configured to detect intra-fraction motion in near-real time using X-ray imaging techniques. Thus, in some embodiments, the RT system 100 may be configured to provide stereotactic radiosurgery and precision radiotherapy for lesions, tumors, and conditions anywhere in the body where radiation treatment is indicated. As such, the RT system 100 can include one or more of a linear accelerator (LINAC) 104 that generates a megavoltage (MV) treatment beam of high energy X-rays or other radiation, one or more kilovolt (kV) X-ray sources 106, one or more imaging panels 107 (e.g., an X-ray imager), and an MV electronic portal imaging device (EPID) 105. By way of example, the RT system 100 is described herein configured with a C-arm gantry 110 capable of infinite rotation via a slip ring connection. In other embodiments, the RT system 100 can be configured with a circular gantry mounted on a drive stand, or any other technically feasible configuration that enables radiation therapy and imaging of a PTV.

In some embodiments, the RT system 100 is capable of X-ray imaging of a target volume immediately prior to and/or during application of an MV treatment beam, so that an image-guided radiation therapy (IGRT) and/or an intensity-modulated radiation therapy (IMRT) process can be performed using X-ray imaging. For example, in some embodiments, the RT system 100 includes kV imaging of a PTV in conjunction with imaging generated by the MV treatment beam. The RT system 100 may include one or more touchscreens (not shown) for patient information verification, motion controls 102, a radiation area 103, a couch positioning assembly 101, a couch 108 disposed on the couch positioning assembly 101, and an image acquisition and treatment control computer 109, all of which are disposed within a treatment room. The RT system 100 further includes a remote control console 111, which is disposed outside the treatment room and enables treatment delivery and patient monitoring from a remote location. In some embodiments, the image acquisition and treatment control computer 109 and/or the remote control console 111 is configured to execute a treatment planning system that includes photon-beam, electron-beam, and/or other treatment planning models that accurately represent the beams generated by the RT system 100. Such models include pre-configured beam data that assumes specific attributes of the beam spot that generates a treatment beam. The couch positioning assembly 101 is configured to precisely position the couch 108 with respect to the radiation area 103, and the motion controls 102 include input devices, such as buttons and/or switches, that enable a user to operate the couch positioning assembly 101 to automatically and precisely position the couch 108 to a predetermined location with respect to the radiation area 103. The motion controls 102 also enable a user to manually position the couch 108 to a predetermined location.

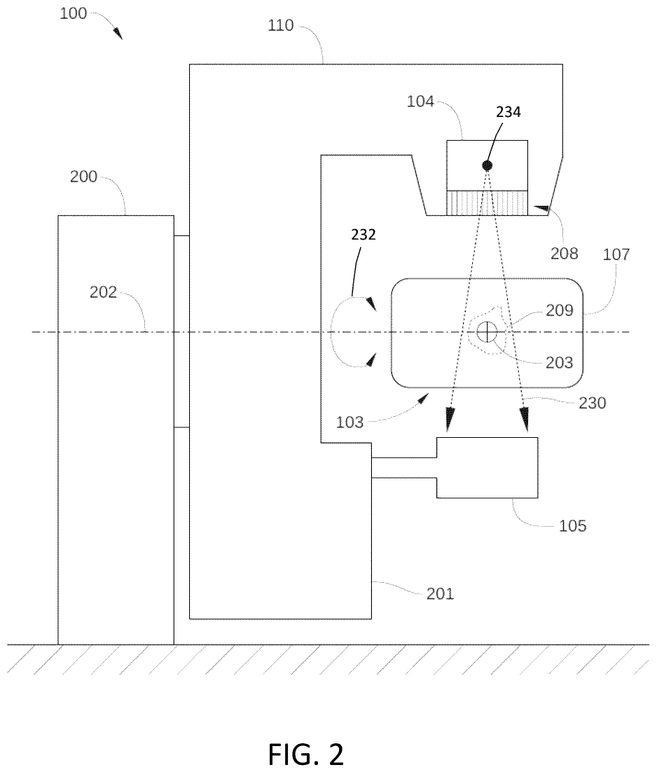

FIG. 2 schematically illustrates a side view of the RT system 100, according to various embodiments. As shown, the RT system 100 includes a base stand 200 and the C-arm gantry 110. In FIG. 2, the couch positioning assembly 101, the couch 108, and the X-ray source 106 are omitted for clarity. The base stand 200 is a fixed support structure for components of the RT system 100, including the C-arm gantry 110 and a drive system (not shown) for rotatably moving the C-arm gantry 110 about a horizontal rotation axis 202, so as to rotationally position the C-arm gantry 110 at one or more gantry angles 232 between 0 and 360 degrees, for example. The base stand 200 rests on and/or is fixed to a support surface that is external to RT system 100, such as a floor of an RT treatment facility. The C-arm gantry 110 is rotationally coupled to the base stand 200 and is a support structure on which various components of the RT system 100 are mounted, including the LINAC 104, the EPID 105, the imaging X-ray source 106 (not shown in FIG. 2), and the imaging panel 107. During operation of the RT system 100 when a treatment beam 230 is being delivered, the C-arm gantry 110 may rotate in a continuous manner about the radiation area 103 when actuated by the drive system or may remain fixed for some amount of time at a particular gantry angle 232—various modalities and variations for delivering the treatment beam 230 by the C-arm gantry 110 at a fixed gantry angle 232 and/or by rotationally moving C-arm gantry 110 to different gantry angles 232 are possible depending on the treatment plan for a patient.

The imaging X-ray source 106 is configured to direct a conical beam of X-rays, referred to herein as imaging X-rays (not shown in FIG. 2 for clarity), through an isocenter 203 of RT system 100 to imaging panel 107. The isocenter 203 typically corresponds to the location of a target (e.g., a target volume 209 to be treated, such as a PTV), with the treatment beam 230 impinging on that location. In the embodiment illustrated in FIG. 2, the imaging panel 107 is depicted as a planar device, whereas in other embodiments, the imaging panel 107 can have a curved configuration. In the embodiment illustrated in FIGS. 1 and 2, the RT system 100 includes a single imaging panel and a single corresponding imaging radiation source in addition to EPID 105. In other embodiments, the RT system 100 can include two or more imaging panels, each with a corresponding imaging radiation source.

The LINAC 104 typically includes one or more of an electron gun for generating electrons, an accelerating waveguide, an electron beam target, an electron beam transport component (such as one or more bending magnets) for directing the electron beam to the electron beam target, and/or a collimator assembly 208 for collimating and shaping the treatment beam 230 that originates from the electron beam target. A source (e.g., a reference point at the LINAC 104) of the treatment beam 230 is symbolically shown at 234 in FIG. 2, and will be explained in further detail later below.

The collimator assembly 208 typically includes one or more of a primary collimator that defines the largest available circular radiation field for the treatment beam 230, a secondary collimator for providing a rectangular or square radiation field at the isocenter 203 (for example via X-jaws and Y-jaws), and a multi-leaf collimator (MLC) for conforming the treatment beam 230 to a PTV or other target volume.

During radiation treatment, in some embodiments, the LINAC 104 is configured to generate the treatment beam 230, which can include high-energy radiation (for example MV X-rays or MV electrons). In other embodiments, the treatment beam 230 includes electrons, protons, and/or other heavy charged particles, ultra-high dose rate X-rays (e.g., for FLASH radiotherapy), and/or microbeams for microbeam radiation therapy. In addition, the imaging panel 107 is configured to receive imaging radiation and generate suitable projection images therefrom. Further, in some embodiments, as the treatment beam 230 is directed to the isocenter 203 while the C-arm gantry 110 rotates through a treatment arc (e.g., gantry angles), image acquisitions can be performed via the EPID 105 to generate image data for the target volume 209. For example, in such embodiments, the EPID 105 generates one or more projection images of the target volume 209 and/or a region of patient anatomy surrounding the target volume 209. Thus, projection images (e.g., 2D X-ray images) of the target volume 209 can be generated during portions of an IGRT or IMRT process via imaging panel 107 and/or the EPID 105. Such projection images can then be employed to construct or update portions of imaging data for a digital volume that corresponds to a three-dimensional (3D) region that includes the target volume 209. That is, a 3D image of such a 3D region is reconstructed from the projection images. In some embodiments, cone-beam computed tomography (CBCT) and/or digital tomosynthesis (DTS) can be used to process the projection images generated by the imaging panel 107.

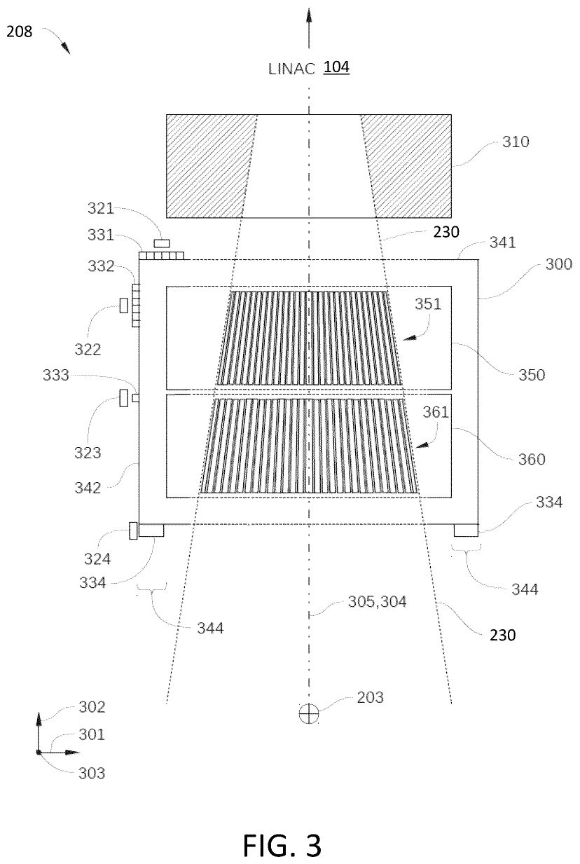

FIG. 3 schematically illustrates the collimator assembly 208 of the RT system 100, according to an embodiment. In the embodiment illustrated in FIG. 3, the collimator assembly 208 includes a primary collimator 310 and an MLC carousel 300 that includes at least one MLC layer. The collimator assembly 208 is disposed proximate the source (not shown) of radiation from the LINAC 104 and between the source and the isocenter 203 of the RT system 100. Further, in some embodiments, the primary collimator 310 may be fixed in position relative to the source, while the MLC carousel 300 is configured to be moved with respect to the source. In some embodiments, the MLC carousel 300 is configured to be translated along one or more linear axes, such as a first axis of linear motion 301, a second axis of linear motion 302, and/or a third axis of linear motion 303 (out of the page). In some embodiments, the MLC carousel 300 is configured to be rotated about at least one axis of rotation, such as an axis of rotation 304. In some embodiments, the axis of rotation 304 is substantially parallel with a central beam axis 305 of the treatment beam 230. In the instance illustrated in FIG. 3, the axis of rotation 304 coincides with central beam axis 305 of the treatment beam 230, but in some situations that will be described later below, the axis of rotation 304 may be displaced from central beam axis 305 along the first axis of linear motion 301 and/or the third axis of motion 303.

In some embodiments, the MLC carousel 300 is configured with primary and secondary position detection for linear motion along the first axis of linear motion 301, the second axis of linear motion 302, the third axis of linear motion 303, and/or the axis of rotation 304. In some embodiments, the primary motion detection with respect to one or more of the above axes is provided by a servo system associated with the motion. For example, in an embodiment, a servo system associated with linear motion of the MLC carousel 300 along the first axis of linear motion 301 includes certain position feedback that indicates the current position of the MLC carousel 300 along the first axis of linear motion 301. In such embodiments, such position feedback is considered primary linear position detection along the axis of linear motion 301. In another example, in an embodiment, a servo system associated with rotational motion of the MLC carousel 300 about the axis of rotation 304 includes certain position feedback that indicates the current rotational position of the MLC carousel 300 about axis of rotation 304. In such embodiments, such rotational position feedback is considered primary rotational position detection.

In some embodiments, motion detection with respect to one or more of the above axes (for example, secondary motion detection) is provided by a respective magnetoresistive sensor. Thus, in such embodiments, the MLC carousel 300 includes one or more of: a magnetoresistive sensor 321 for motion detection of the MLC carousel 300 with respect to the first axis of linear motion 301; a magnetoresistive sensor 322 for motion detection of the MLC carousel 300 with respect to the second axis of linear motion 302, a magnetoresistive sensor 323 for motion detection of the MLC carousel 300 with respect to the third axis of linear motion 303, or a magnetoresistive sensor 324 for motion detection of the MLC carousel 300 with respect to the axis of rotation 304. In such embodiments, the magnetoresistive sensor 321 performs motion detection via a linear array 331 of magnets disposed on a surface 341 of the MLC carousel 300, the magnetoresistive sensor 322 performs motion detection via a linear array 332 of magnets disposed on a surface 342 of the MLC carousel 300, magnetoresistive sensor 323 performs motion detection via a linear array 333 of magnets disposed on surface 342 of the MLC carousel 300, and/or magnetoresistive sensor 324 performs motion detection via a toothed ring 334 disposed on a peripheral region 344 of the MLC carousel 300. In such embodiments, an International Electrotechnical Commission (IEC) requirement for a secondary position sensor is for all LINAC carousel linear and rotational axes can be satisfied by a respective magnetoresistive sensor.

The primary collimator 310 may be configured to define an outer limit of the field of the treatment beam 230. The primary collimator 310 can be a fixed collimator or a collimator configured with one or more movable jaws. Typically, the primary collimator 310 is disposed proximate the radiation source of the LINAC 104. In the embodiment illustrated in FIG. 3, the primary collimator 310 is depicted as a single collimating apparatus, but in other embodiments, the primary collimator 310 includes multiple collimating apparatuses positioned in series within the field of the treatment beam 230.

In some embodiments, the MLC carousel 300 includes a proximal MLC layer 350 and a distal MLC layer 360. In other embodiments, the MLC carousel 300 includes a single MLC layer. The proximal MLC layer 350 includes a plurality of leaves 351 that are each independently movable into the travel direction of the treatment beam 230. Similarly, the distal MLC layer 360 includes a plurality of leaves 361 that are each independently movable into the travel direction of the treatment beam 230. In the embodiment illustrated in FIG. 3, each leaf 351 of the proximal MLC layer 350 is movable in one particular travel direction, which is perpendicular to the central beam axis 305 of the treatment beam 230. Further, in the embodiment illustrated in FIG. 3, the travel direction of the leaves 351 is depicted to be along third axis of linear motion 303, which is out of the page. Similarly, each leaf 361 of the distal MLC layer 360 is movable in one particular travel direction that is perpendicular to central beam axis 305 of the treatment beam 230. In the embodiment illustrated in FIG. 3, the travel direction of the leaves 361 is the same travel direction as that of the leaves 351, which is along the axis of linear motion 303. In FIG. 3, the leaves 351 and the leaves 361 are viewed end-on, i.e., along the travel direction, which is parallel to the third axis of linear motion 303.

In some embodiments, the proximal MLC layer 350 includes multiple banks of leaves 351 and the distal MLC layer 360 includes multiple banks of leaves 361. In such embodiments, the MLC layer 350 includes two opposing banks of leaves 351 that are positioned on opposite sides of a center plane of the treatment beam 230, and distal MLC layer 360 includes two opposing banks of leaves 361 that are positioned on opposite sides of the center plane of the treatment beam 230.

The leaves 351 and 361 are typically formed from a high atomic number material, such as tungsten or an alloy thereof. In addition, in some embodiments, the leaves 351 and 361 have a generally trapezoidal cross-section that matches the beam divergence that occurs in the direction perpendicular to leaf travel. In practice, the cross-section of the leaves 351 and 361 may not be exactly trapezoidal, and may have other shapes such as rectangular, other polygonal shape, or other shape. In some embodiments, the leaves 351 and leaves 361 may be configured to project to a same projected size at the isocenter 203. In such embodiments, the leaves 351 have a smaller cross-section in the direction perpendicular to leaf travel than the leaves 361.

In some embodiments, motion detection of each of the leaves 351 and leaves 361 along a direction of linear travel is enabled by a respective magnetoresistive sensor. In such embodiments, each leaf 351 and each leaf 361 includes a magnetoresistive sensor for linear motion detection of the corresponding leaf.

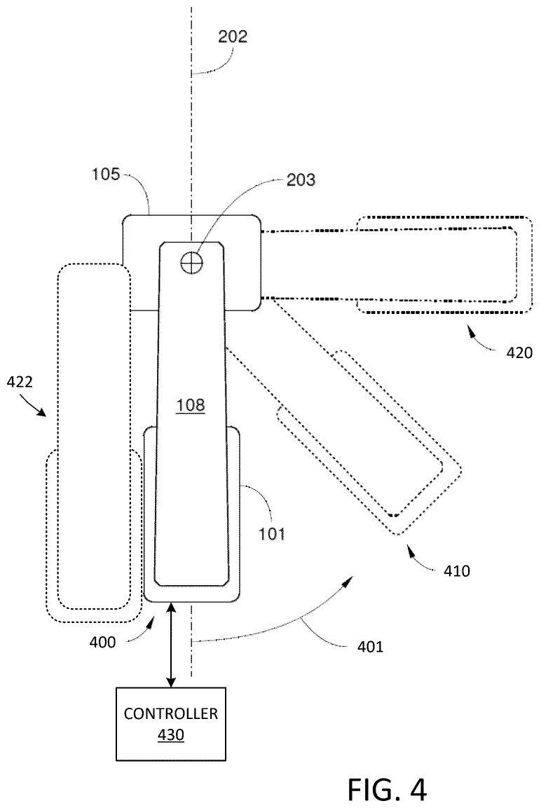

With reference now to the couch 108 and according to various embodiments, the couch positioning assembly 101 is configured to rotate, pitch, roll, and/or translate couch 108 relative to the isocenter 203 to one or more treatment positions. One such example embodiment is described below in conjunction with FIG. 4.

FIG. 4 schematically illustrates plan views of the couch 108 in various treatment positions, according to various embodiments. FIG. 4 includes a plan view of the couch 108 in a neutral position 400, in which the couch 108 is in line with the horizontal rotation axis 202 of the C-arm gantry 110, a first rotated position 410 (dashed lines), in which the couch 108 is rotated 45 degrees from the neutral position 400, and a second rotated position 420 (dashed lines), in which the couch 108 is rotated 90 degrees from the neutral position 400. For reference, the EPID 105 and the isocenter 203 are both included in FIG. 4. As shown in the example of FIG. 4, the couch positioning assembly 101 rotates the couch 108 about the isocenter 203 to a couch rotational angle 401 from the neutral position 400. The couch rotational angle 401 can be, for example, up to about 90 degrees.

The above-described illustrated example in FIG. 4 involves the couch 108 being rotated about the isocenter 203. In other embodiments described later below, the couch positioning assembly 101 may move the couch 108 to other positions relative to the isocenter 203 and/or the treatment beam 230, such as laterally along a plane (e.g., along an X-Y plane) so as to be closer to, further away from, or in coincidence with the isocenter 203 and/or with the central beam axis 305 of the treatment beam 230. For instance, at a position 422, the couch 108 has been moved laterally further away from the isocenter 203 and the treatment beam 230 (e.g., to the left and down on the page).

According to various embodiments, the couch positioning assembly 101 of FIG. 1 can be used to perform the foregoing adaptive positioning of the couch 108, per gantry angle. A couch position controller 430, which may be part of or operatively coupled to the couch positioning assembly 101, the motion controls 102, the remote control console 111, and/or other device(s), may be used to control the positioning of the couch 108. According to various embodiments described later below, the couch position controller 430 may work in conjunction with the couch positioning assembly 101 to perform the adaptive positioning of the couch 108 relative to the treatment beam 230 (and its central beam axis 305), per gantry angle.

Alternatively or additionally, the couch positioning assembly 101 may move the couch 108 in raised/lowered direction (e.g., along a Z-axis) relative to the isocenter 203 and/or the treatment beam 230. It is also possible in some embodiments for the couch positioning assembly 101 to move the couch 108 in a rotational manner (e.g., tilt) along a horizontal axis. As such, the couch positioning assembly 101 is configured to move the couch 108 to any position (e.g., location, orientation, etc.) relative to the isocenter 203 and/or the central beam axis 305 of the treatment beam 230, in accordance with a treatment plan and/or based on other considerations/factors.

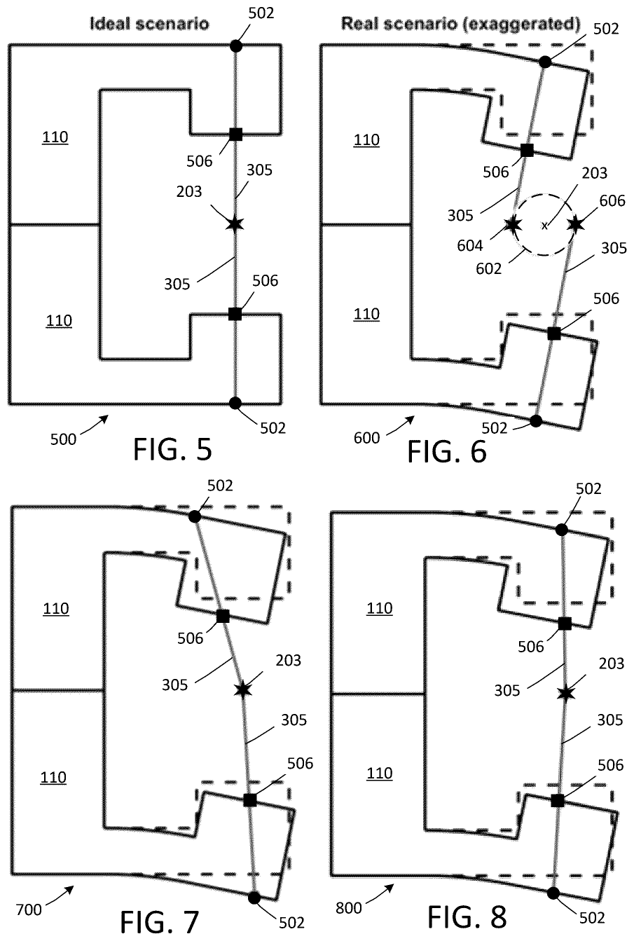

Examples of such considerations/factors, which may be used in some embodiments to adjust or otherwise change the position of the couch 108 and/or the position of the central beam axis 305 of the treatment beam 230, are depicted in FIGS. 5 and 6. More specifically, FIGS. 5 and 6 are schematic side views of the C-arm gantry 110 of the RT system 100 of FIG. 1, in accordance with some embodiments.

In FIG. 5 (and also in subsequent figures), the C-arm gantry 110 is shown at two gantry angles (positions), such as at a gantry angle of 0 degrees (gantry head up) in the upper part of FIG. 5 and at a gantry angle of 180 degrees (gantry head down) in the lower part of FIG. 5. FIG. 5 illustrates an ideal scenario 500 in which the C-arm gantry 110 extends in a substantially horizontal manner, while positioned at both gantry angle 0 degrees and 180 degrees (and at any gantry angle between 0 and 360 degrees).

The point that minimizes the distance to all central beam axes 305 of treatment beams 230 at all gantry angles is the isocenter 203 (depicted as a solid star in FIG. 5), and that is the location where a target (e.g., a tumor) is often placed during a treatment regimen using image-based techniques (MV/kV matching CBCT, etc.). A maximum distance from the isocenter 203 to the furthest central beam axis 305 is the isocenter size 602 (shown in FIG. 6 as a larger isocenter size 602 relative to FIG. 5 in which the isocenter size is minimized to zero). The isocenter size can be interpreted as a maximum delivery error, and so reduction of the isocenter size (ideally to near zero) would be advantageous in order to more effectively and precisely deliver the treatment beam 230 to a target area that may be of small size.

According to various embodiments and as depicted in FIG. 5 (and in subsequent figures), the central beam axis 305 is the line defined by two points: a source 502 (at the LINAC 104, such as at 234 in FIG. 2) of the treatment beam 230, and the field center 506 of a collimation device (e.g., the collimator assembly 208 having one or more MLCs). Further details of the source 502 and the field center 506 will be provided later below.

In the ideal scenario 500 of FIG, 5, all central beam axes 305 are tuned to (e.g., impinge upon) the same location, at the isocenter 203 (e.g., isocenter size is zero), due to the uniform horizontal extension of the C-arm gantry 110 at all gantry angles. However, the ideal scenario 500 is often not typical.

FIG. 6 depicts a more common scenario 600, in which the C-arm gantry 110 has a mechanical deformation (shown in an exaggerated manner in FIG. 6 and in subsequent figures for purposes of clarity and emphasis). More specifically, the C-arm gantry 110 has a gantry head that bends/sags downward due to gravity. This downward deformation may be due to factors affected by gravity such as weight of the C-arm gantry 110, its age, wear and tear through usage, etc. In some cases, the downward deformation may be present in a newly manufactured C-arm gantry 110, as a result of mechanical tolerances, minor defects, etc.

As depicted in FIG. 6, the alignment of the central beam axis 305 with respect to a target position/location for the treatment beam 230 can change with gantry angle. For the gantry angle of 0 degrees (gantry head up) as an example, the central beam axis 305 is misaligned, such that the treatment beam 230 impinges at a location 604 that is offset or deviates from the ideal isocenter 203. For the gantry angle of 180 degrees (gantry head down) as another example, the central beam axis 305 is misaligned, such that the treatment beam 230 impinges at a location 606 that is also offset or deviates from the ideal isocenter 203. A result is the larger isocenter size 602, relative to the smaller isocenter size (zero) of FIG. 5. One solution to address the deformation of a C-arm gantry is to build more robust arms that do not have large deformations and therefore minimize the isocenter size—however, such a solution is often impractical.

FIG. 7 is a schematic side view of the C-arm gantry 110 of the radiation therapy system 100 of FIG. 1 in which a position of the source 502 is adjusted, according to various embodiments. For the gantry angle of 0 degrees as an example, the position of the source 502 is moved to the left. For the gantry angle of 180 degrees as another example, the position of the source 502 is moved to the right. Since the central beam axis 305 is defined by a line through two points (e.g., the adjusted position of the source 502 and the field center 506), the central beam axes 305 is tuned (for all gantry angles) by appropriately changing/adjusting the position of the source 502 (at all gantry angles), such that all central beam axes 305 impinge upon the same isocenter 203. Explained in another way, the changed position of the source 502 realigns the central beam axis 305, for each of the gantry angles, to the same isocenter 203 where the target is placed. Thus, with this elimination of (or compensation for) the deviation of the central beam axes 305 from the isocenter 203, the size of the isocenter 203 is reduced (ideally to near zero).

According to some embodiments, the position of the source 502 may be adjusted by changing the magnetic field strength of one or more bending magnets (e.g., one or more electromagnets). An adaptive source position controller may be provided in the RT system 100 for adjusting the magnetic field strength of the bending magnet(s) per gantry angle.

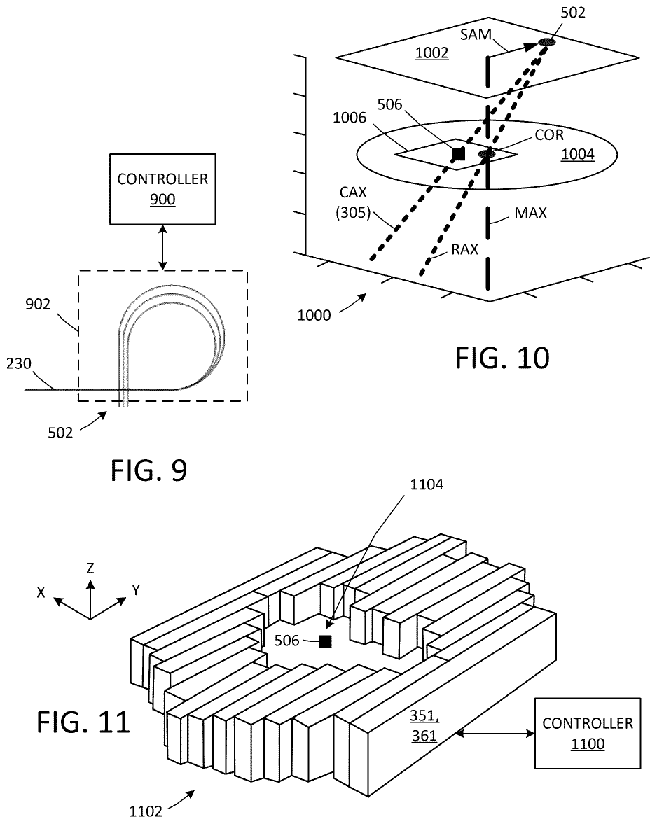

More specifically and by way of example, FIG. 9 schematically illustrates an example of an adaptive adjustment of the position of the source 502, according to various embodiments. In the LINAC 104, the treatment beam 230 is generated by an electron gun (not shown) and propagates through a waveguide (not shown). One or more bending magnets 902 (symbolically represented by a dashed box) apply magnetic field(s) to the treatment beam 230, such that the trajectory (path) of the treatment beam 230 is bent. In the example of FIG. 9, the path of the treatment beam 230 is bent approximately 90 degrees by the bending magnet(s) 902, which may be electromagnets.

According to various embodiments, an adaptive source position controller 900 may be used to adjust/change the strength of the magnetic field(s) of the bending magnet(s) 902. For example, the controller 900 may increase/decrease the strength of the magnetic field(s) in order to control the amount of bending (and hence the direction) of the treatment beam 230. In the example of FIG. 9, three different directions of the treatment beam 230 are shown, each based upon an appropriate adjustment of the magnetic field strength.

The source 502 may be, in some embodiments, any appropriate reference point in the LINAC 104 along the central beam axis 305 of the treatment beam 230, after the treatment beam 230 has been bent by the magnet(s) 902. Hence in the example of FIG. 9, there may be three different positions of the source 502 for the particular gantry angle, each corresponding to a differently bent treatment beam 230. In other embodiments, the source 502 may be some other reference point inside or outside of the LINAC 104, along the central beam axis 305 and before reaching the field center 506 of the collimator assembly 208. The magnetic field(s) of the bending magnet(s) 902 may be adjusted in the manner depicted in FIG. 9 by the controller 900 per gantry angle, thereby providing adaptive adjustment to multiple positions of the source 502 for any/each gantry angle between 0 and 360 degrees.

With reference now to FIG. 8, FIG. 8 is a schematic side view of the C-arm gantry 110 of the radiation therapy system 100 of FIG. 1 in which a position of a field center of a collimation assembly is adjusted, according to various embodiments. More specifically and in comparison to the embodiment of FIG. 7, the embodiment of FIG. 8 adjusts the position of the field center 506 of the collimator assembly 208, or more particularly, the field center 506 of at least one MLC layer (e.g., the MLC layer 350 and/or 360 of FIG. 3).

The position of the field center 506 may be adjusted in various embodiments by using an MLC controller. Typically, a position of a field center is adjusted such that the field center is aligned to the axis of rotation of a collimator. However, with the embodiment illustrated in FIG. 9, a compensation component is added to the MLC controller, wherein the corrections/adjustments to the field center 506 are applied per gantry angle. With this adjustment method, all central beam axes 305 are tuned to the isocenter 203, thereby reducing the isocenter size 602 (ideally to zero).

In FIG. 8, for the gantry angle of 0 degrees as an example, the position of the field center 506 is moved to the right. For the gantry angle of 180 degrees as another example, the position of the field center 506 is moved to the left. Since the central beam axis 305 is defined by a line through two points (e.g., the unchanged position of the source 502 and adjusted position of the field center 506), the central beam axes 305 can be tuned (for all gantry angles) by appropriately changing/adjusting the position of the field center 506 (at all gantry angles), such that all central beam axes 305 impinge upon the same isocenter 203.

FIG. 11 is a perspective view of an example multi-leaf collimator (MLC) 1102 having an adjustable field center, according to various embodiments. For instance, the MLC 1102 of FIG. 11 may represent at least one MLC layer (e.g., the MLC layers 350 or 360) in the collimator assembly 208 of FIG. 3. The MLC 1102 may include a plurality of the adjustable leaves 351/361 as previously described.

An MLC controller 1100 is operatively coupled to the MLC 1102 and is configured to control/adjust the respective position of each individual leaf 351/361, so as to provide an aperture 1104 through which the treatment beam 230 passes. The size, shape, location, etc. of the aperture 1104 may be adjusted by the controller 1100 dependent on factors such as the parameters of the treatment regimen, the size, shape, and location of the target area or isocenter, etc. The field center 506 of the MLC 1102 lies within the aperture 1104.

To provide adjustment of the position of the field center 506, such as depicted in the example of FIG. 8, the controller 1100 is configured to shift/move the entirety of the leaves 351/361 along the X-axis and/or Y-axis shown in FIG. 11. Thus, moving all of the leaves 351/361 as an entire group in this manner results in a corresponding adjustment in the location of the field center 506 and the aperture 1104 defined by the leaves. It is noted that the controller 1100 may also move the leaves 351/361 along the Z-axis (e.g., up or down) or rotate the leaves 351/361 about the Z-axis (e.g., an axis of rotation).

In some embodiments, the adjustment of the position of the field center 506 may be performed by selectively repositioning individual leaves 351/361, rather than by collectively moving all of the leaves along the X-axis or Y-axis. For instance in FIG. 11, the placement of individual leaves 351/361 can be shifted along the Y-axis by the controller 1110, so as to correspondingly redefine the location/position (as well as the shape, size, etc.) of the aperture 1104. This redefinition of the location/position of the aperture 1104 results in a corresponding change in location/position of the field center 506, along the X-axis and/or the Y-axis.

To further explain and illustrate the source 502 and the field center 506 in the context of adjusting their respective positions for purposes of tuning the treatment beam 230 to the isocenter 203, reference is made next to FIG. 10. FIG. 10 is a diagram 1000 illustrating relationships between a position of the source 502 and a position of the field center 506, according to various embodiments. The source 502 lies on a source plane 1002, and the field center 506 lies on a collimator plane 1004.

The following aspects are illustrated in FIG. 11:
MAX: the mechanical axis of rotation (AOR) of a collimator;
SAM: the source-axis misalignment, which is the deviation of the source 502 with respect to the collimator AOR (MAX), such as when the position of the source 502 is changed in the manner previously described above;
RAX: the radiation axis, which is a line passing through the source 502 and the center of rotation (COR) of a collimation element (e.g., the proximal MLC layer 350, the proximal MLC layer 360, the jaws of the primary collimator 310, etc.). Note that when the SAM is not equal to zero, each collimation element may have its own RAX; and
CAX: the central beam axis 305, which is shown as a line passing through the source 502 and the field center 506. A shape of a collimation field 1006 (such as provided by the aperture 1104) having the field center 506 may be in the shape of a square, circle, or other shape(s). In the case of an ideal collimator, the CAX is coincident with the RAX. In the case of a non-ideal collimator, each gantry angle may have multiple CAX corresponding to the collimator rotation.

Movement of the source 502 along the source plane 1002 (as depicted by the SAM in FIG. 10) corresponds to changing the source position as described previously above with respect to FIGS. 6, 7, and 9. Movement of the field center 506, from the COR to some other position in the collimation plane 1004 as depicted in FIG. 10, corresponds to changing the position of the field center 506 as described previously above with respect to FIGS. 5, 8, and 11.

The foregoing embodiments involve adjustment of the position of the source 502 and the field center 506 so as to reduce the size of the isocenter 203. In such embodiments, a target (e.g., a tumor) can be kept aligned with the treatment beam 230 such that the treatment beam 230 impinges upon the target at the isocenter 203. Other techniques may be used to align or otherwise position a target relative to the treatment beam 230, and will be described next below.

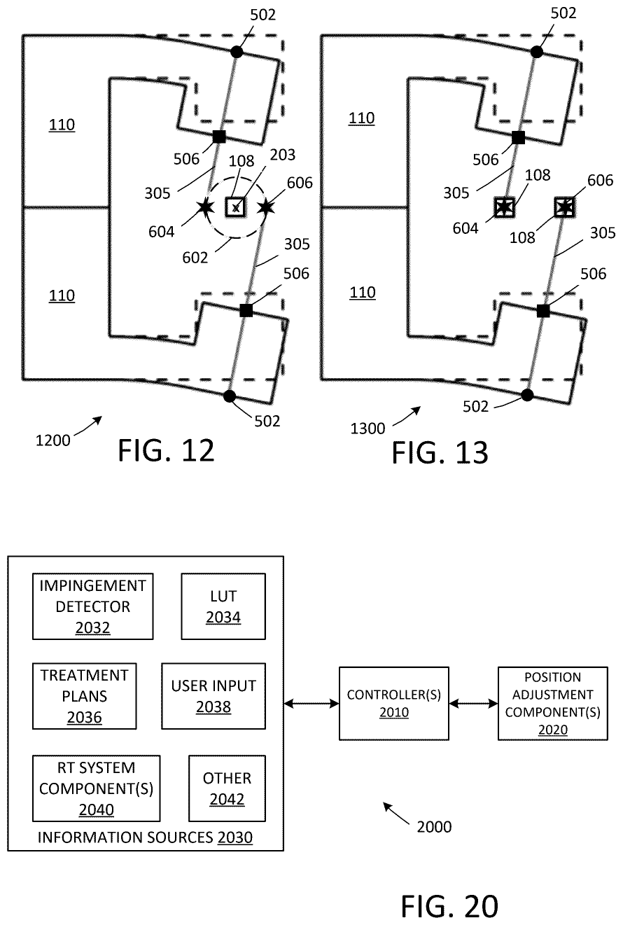

FIG. 12 is schematic side view of the C-arm gantry 110 of the radiation therapy system 100 of FIG. 1 illustrating positioning of the couch 108 relative to a treatment beam 230 (e.g., the central beam axis 305), according to various embodiments. As previously described with respect to FIG. 6 (and using similar labeling as in FIG. 6), a scenario 1200 in FIG. 12 shows that for the gantry angle of 0 degrees (gantry head up) as an example, the central beam axis 305 is misaligned, such that the treatment beam 230 impinges at a location 604 that is offset from the ideal isocenter 203. For the gantry angle of 180 degrees (gantry head down) as another example, the central beam axis 305 is misaligned, such that the treatment beam 230 impinges at a location 606 that is also offset from the ideal isocenter 203. A result is the larger isocenter size 602.

In the scenario 1200 of FIG. 12, the couch 108 is placed in a fixed position such that the target (e.g., a tumor on a patient lying on the couch 108) is positioned between locations 604 and 606, at the ideal isocenter 203. Therefore, in the scenario 1200, there may not be a central beam axis 305 that directly impinges on or coincides with the target (e.g., the target is misaligned with or is offset from the treatment beam 230), thereby possibly reducing the effectiveness of the treatment.

To address the foregoing misalignment, various embodiments use an adaptive couch position, where the positioning of the couch 108 compensates for the offset of the central beam axis 305 and keeps the target aligned to the treatment beam 230. This adaptive couch positioning can be performed for static gantry fields (e.g., single offset compensation) but also for volumetric modulated arc therapy (VMAT) treatments (continuous offset compensation) and/or other implementations.

FIG. 13 illustrates examples of the adaptive couch positioning for a scenario 1300. More specifically, FIG. 13 is a schematic side view of the C-arm gantry 110 of the radiation therapy system 100 of FIG. 1 illustrating positioning of the couch 108 relative to the treatment beam 230 (e.g., the central beam axis 305), according to various embodiments. The scenario 1300 in FIG. 13 shows that for the gantry angle of 0 degrees (gantry head up) as an example, the couch 108 is moved to the left such that the target is coincident with (e.g., is impinged upon by) the central beam axis 305 at the location 604. For the gantry angle of 180 degrees (gantry head down) as another example, the couch 108 is moved to the right such that the target is coincident with (e.g., is impinged upon by) the central beam axis 305 at the location 606.

Thus, adaptively changing the position of the couch 108 compensates for any offset from the central beam axis 305. This adaptive positioning of the couch 108 therefore always keeps the target aligned to the central beam axis 305 so as to achieve a more accurate delivery of the treatment.

The preceding description with respect to FIG. 13 involves the scenario 1300 in which the target aligned to the central beam axis 305 such that the target coincides with the central beam axis 305. As previously explained above, the isocenter 203 is defined as a point that minimizes the distances to all central beam axes 305. However, not all central beam axes 305 might have the same importance overall, and moreover, might not have the same importance for each individual treatment plan.

Accordingly, other embodiments use an adaptive optimal isocenter according to weights that are assigned to each central beam axis 305. A weighting strategy of the central beam axes 305 can be defined in a treatment plan. The following are non-exhaustive examples of weighting strategies/assignments:
Infinite weight to all central beam axes 305 (minimizes distance to any central beam axis 305, prioritizes avoiding an organ at risk (OAR);
Uniform weight to all (minimization of least-square distances, prioritizes hitting the target);
User-defined weight for each field (treatment beam 305/central beam axis 305);
Percentage of monitor unit (MU) delivered along each central beam axes 305;
According to a robustness metric defined in the treatment plan system which defines how robust is that field to geometric uncertainties; or
Other weighting assignment/method including combinations thereof.

Patient positioning, or more specifically, positioning of the couch 108 (on which the target lies) is performed by aligning the couch 108 to one or more central beam axes 305 in accordance with the weight(s) assigned to the central beam axes 305. In embodiments that use such weighting, the isocenter size becomes a less relevant metric, as the weights can increase the maximum distance from the optimal isocenter to the furthest central beam axis 305.

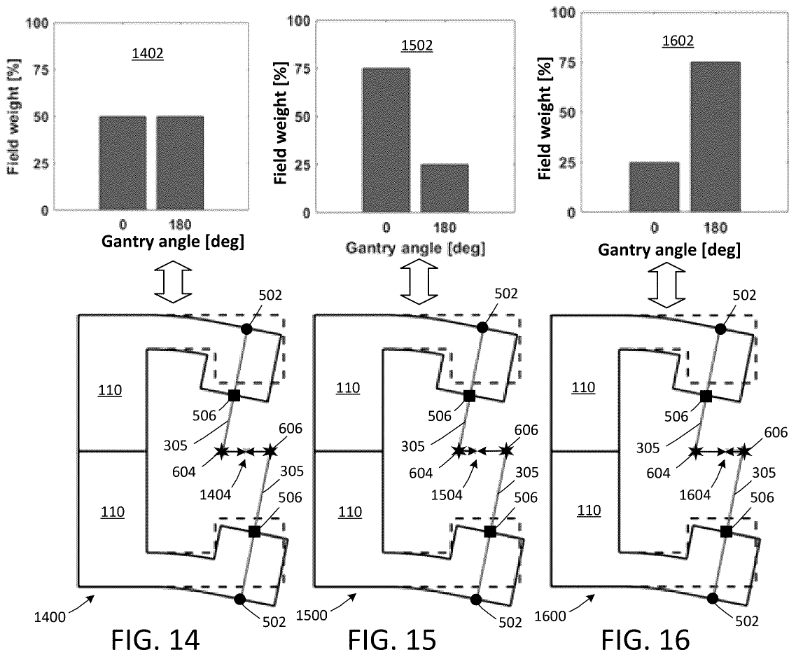

Examples are described next below with respect to embodiments in which the position of the couch 108 is aligned with (but not necessarily coincident with) one or more central beam axes 305 of treatment beam(s) 230 or fields. FIGS. 14, 15, 16, 17, 18, and 19 are schematic side views of the C-arm gantry of the radiation therapy system of FIG. 1 illustrating positioning of a couch relative to treatment beams based on weights, according to various embodiments.

FIGS. 14, 15, and 16 illustrate example weighting of fields (e.g., the treatment beam 230 and its central beam axis 305) at two gantry angles. Referring first with respect to FIG. 14 and as previously described with respect to FIG. 6 (and using similar labeling as in FIG. 6), a scenario 1400 in FIG. 14 shows that for the gantry angle of 0 degrees (gantry head up) as an example, the central beam axis 305 is misaligned, such that the treatment beam 230 impinges at a location 604 that is offset from (e.g., deviates from) an ideal isocenter (see, e.g., the ideal isocenter 203 in FIG. 5 ad 6). For the gantry angle of 180 degrees (gantry head down) as another example, the central beam axis 305 is misaligned, such that the treatment beam 230 impinges at a location 606 that is also offset (e.g., deviates from) from the ideal isocenter. The positions of the source 502 and the field center 506 remain unchanged.

A corresponding graph 1402 shows a weighting strategy that has been defined for a treatment plan. In this weighting strategy, the field weight is 50% at both gantry angles 0 degrees and 180 degrees. With this equal 50% weighting for the fields at these two gantry angles, the couch 108 may be positioned (e.g., by the controller 430 of FIG. 4) at a location 1404, so that the target is positioned (depicted in FIG. 14 by arrows) between (midway) the locations 604 and 606. Explained in another way, while the target is aligned with both treatment beams 230, the target is not placed in coincidence with the central axis 305 of a first treatment beam 230 at gantry angle 0 degrees or with the central axis 305 of a second treatment beam 230 at gantry angle 180 degrees, but is instead offset/distanced from these central beam axes 305 based on the assigned weighting.

Referring next to FIG. 15, a corresponding graph 1502 shows a weighting strategy that has been defined for a treatment plan. In this weighting strategy, the field weight is 75% at gantry angle 0 degrees and is 25% at gantry angle 180 degrees. Thus, the treatment beam 230 (central beam axis 305) at gantry angle 0 degrees (gantry head up) is higher weighted than the treatment beam 230 (central beam axis 305) at gantry angle 180 degrees (gantry head down).

With this higher weighting at gantry angle 0 degrees, FIG. 15 shows that the couch 108 may be positioned (e.g., by the controller 430 of FIG. 4) at location 1504, so that the target is positioned (depicted in FIG. 15 by arrows) closer to the location 604 and further away from the location 606. Again, while the target is aligned with both treatment beams 230 so as to achieve the weighting assignment, the target is not placed in coincidence with the central axis 305 of a first treatment beam 230 at gantry angle 0 degrees or with the central axis 305 of a second treatment beam 230 at gantry angle 180 degrees, but is instead offset/distanced from these central beam axes 305 based on the assigned weighting. It is noted that the couch 108 (target) could be placed in coincidence with the central beam axis 305, at the location 604, if the assigned weight for gantry angle 0 degrees is 100%, for example.

Referring next to FIG. 16, a corresponding graph 1602 shows a weighting strategy that has been defined for a treatment plan. In this weighting strategy, the field weight is 25% at gantry angle 0 degrees and is 75% at gantry angle 180 degrees. Thus, the treatment beam 230 (central beam axis 305) at gantry angle 0 degrees (gantry head up) is lower weighted than the treatment beam 230 (central beam axis 305) at gantry angle 180 degrees (gantry head down).

With this higher weighting at gantry angle 180 degrees, FIG. 16 shows that the couch 108 may be positioned (e.g., by the controller 430 of FIG. 4) at location 1604, so that the target is positioned (depicted in FIG. 16 by arrows) closer to the location 606 and further away from the location 604.

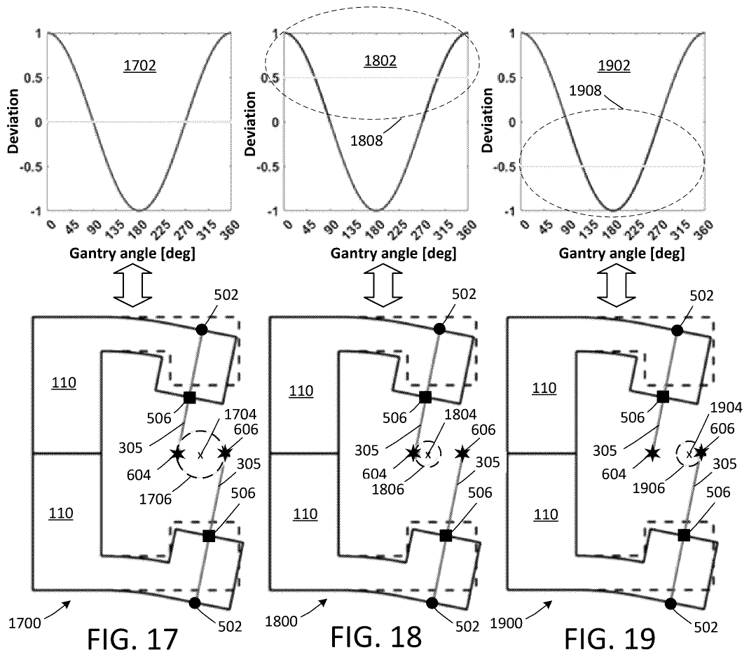

FIGS. 17, 18, and 19 illustrate example weighting based on deviation of the central beam axis 305 from the isocenter 203. In these examples, it is assumed that the deviation of the central beam axis 305 from the isocenter 203 behaves like a cosine function. This may be typical for a LINAC with the C-arm gantry, where the gravity bends the gantry head towards the base stand when the gantry head is up, and away from base stand when the gantry head is down.

According to the examples of FIGS. 17, 18, and 19, the isocenter position and the isocenter size can be defined for three different treatment plans. With reference first to FIG. 17, a scenario 1700 represents a treatment plan in which all central beam axes 305 are considered. A corresponding graph 1702 shows the cosine function for the deviation of the central beam axes 305 from an isocenter 1704 (e.g., the ideal isocenter 203). Since the treatment plan specifies that all central beam axes 305 are considered, an isocenter size 1706 is bounded by the locations 604 and 606 and other locations corresponding to deviations of the treatment beam 230 from the isocenter 1704 for other gantry angles. The couch 108 can be positioned such that the target is placed within the bounds of the isocenter size 1706.

Referring next to FIG. 18, a scenario 1800 represents a treatment plan in which only the central beam axes 305 corresponding to a top semi-sphere 1808 of a graph 1802 are considered. The top semi-sphere 1808 encompasses gantry angles 0 degrees to 90 degrees and 270 degrees to 360 degrees, for example. An isocenter is thus defined/located at 1804 and which has a smaller isocenter size 1806 (relative to what is shown in FIG. 17) for gantry angle 0 degrees and the other gantry angles corresponding to the top semi-sphere 1808. As shown in FIG. 18, the isocenter size 1806 is bounded by the location 604 (corresponding to the deviation at gantry angle 0 degrees) and by other locations corresponding to the selected deviations shown in the graph 1802. The couch 108 can be positioned such that the target is placed within the bounds of the isocenter size 1806.

Referring next to FIG. 19, a scenario 1900 represents a treatment plan in which only the central beam axes 305 corresponding to a bottom semi-sphere 1908 of a graph 1902 are considered. The bottom semi-sphere 1908 encompasses gantry angles 90 degrees to 180 degrees and 180 degrees to 270 degrees, for example. An isocenter is thus defined/located at 1904 and which has a smaller isocenter size 1906 (relative to what is shown in FIG. 17) for gantry angle 180 degrees and the other gantry angles corresponding to the bottom semi-sphere 1908. As shown in FIG. 19, the isocenter size 1906 is bounded by the location 606 (corresponding to the deviation at gantry angle 180 degrees) and by other locations corresponding to the selected deviations shown in the graph 1902. The couch 108 can be positioned such that the target is placed within the bounds of the isocenter size 1906.

Hence, the foregoing examples illustrate that the various embodiments enable the target to be aligned to an isocenter that is optimized to the individual treatment plan, rather than a global isocenter that is assumed to be valid or common for all patients. Such embodiments reduce the optimal isocenter size, and since the isocenter size is the maximum delivery error, a more accurate geometric dose delivery to a patient is achieved.

According to various embodiments, treatment plan-dependent positioning of the target (such as described above with respect to the examples of FIGS. 14, 15, 16, 17, 18, and 19) can involve static positioning, in that when the controller 430 moves the couch 108 to a specific position (so as to place the target at a desired location), the position of the couch 108 and/or the target remains static (e.g., is not changed) during the course of the treatment. This static positioning can be contrasted/compared to the positioning of the source 502, the field center 506 of an MLC, or the couch 108 (such as previously described above with respect to the examples of FIGS. 7, 8, 9, 10, 11, 12, and 13) in which the positioning of the source 502, the field center 506 of an MLC, or the couch 108 can be dynamically changed during the course of the treatment. For instance in the examples of FIGS. 7, 8, 9, 10, 11, 12, and 13, the gantry 110 may rotate to different gantry angles during a treatment session, and the position of the source 502, the field center 506 of an MLC, or the couch 108 can be correspondingly changed so as to keep the target aligned (e.g., coincident) with each central beam axis 305 for each gantry angle.

FIG. 20 is a block diagram illustrating example components in the RT system 100 of FIG. 1 that may be used to implement positional adjustment, according to various embodiments. For instance, the example components may be part of a subsystem 2000 of the RT system 100.

The subsystem 2000 includes at least one controller 2010, at least one position adjustment component 2020, and information sources 2030. The controller(s) 2010, position adjustment component(s) 2020, and information sources 2030 may be communicatively coupled to each other.

The controller(s) 2010 may include the couch position controller 430 of FIG. 4, the source position controller 900 of FIG. 9, the MLC controller 1100 of FIG. 11, and/or other controllers or analogous components in the RT system 100. The controller(s) 2010 may be implemented in some embodiments by a computing device or related subcomponents thereof (such as by a processor configured to execute instructions). The controller(s) 2010 are configured to send, receive, or otherwise process information to/from the information sources 2030, as well as being configured to perform/control positional changes in the manner described herein.

The information source 2030 may include an impingement detector 2032 configured to determine the location of where the central beam axis 305 of the treatment beam 230 impinges on a target. For example, the impingement detector 2032 may determine the amount and location of a deviation/misalignment/offset of a central beam axis 305 relative to an ideal isocenter 203 (e.g., an isocenter with an isocenter size of zero) for each gantry angle. For instance, the impingement detector 2032 can measure the amount (e.g., in millimeters or other unit of length) that the central beam axis 305 deviates from the isocenter 203, per gantry angle, and the controller(s) 2010 can store such measurements in a lookup table (LUT) 2034.

Various tools/techniques may be used for the impingement detector 2032 to detect and measure where the treatment beam 230 impinges the radiation area 103. Examples include optical cameras, water tank equipment, computer-performed detection and measurement algorithms, or other detection/measurement tools/techniques.

The controller(s) 2010 may store the measurements or other information from the impingement detector 2032 in the LUT 2034. For example, the LUT 2034 may have tables or other data structures that relate or otherwise contain: the amount of deviation/misalignment/offset of each central beam axis 305 for each respective gantry angle relative to the isocenter 203; the amount of positional adjustment needed for the source 502, field center 506, or couch 108 in order to reduce isocenter size, for each gantry angle; the deviated/misaligned/offset location (e.g., in X, Y, and/or Z coordinates) of where the central beam axis 305 impinges on a target for each gantry angle, and so forth. According to various embodiments, the information for the LUT 2034 can be obtained and stored during a calibration phase and/or during real-time operation of the RT system 100.

The information sources 2030 may further include the treatment plans 2036 for each patient. As previously explained above, each treatment plan 2036 may specify, among other things, the weights assigned to each central beam axis 305 or other weighting strategy. In some embodiments, at least some of the information (e.g., weighting strategy) of the treatment plans 2036 may be contained in the LUT 2034, so as to relate the weighting strategy to positional adjustment information for the couch 108.

The information sources 2030 may include user input 2038, such as customized settings for the RT system 100 or other information provided by a user that pertains to operation of the RT system 100. Such information from the user input 2038 can be stored in the LUT 2034 in some embodiments, such as during the calibration phase and/or during real-time operation of the RT system 100.

The information sources 2030 may also information that pertain to one or more RT system components 2040, such as the settings (e.g., default and current) for the position of the source 502, the strength of the magnet(s) 902, the position (e.g., default and current) for the field center 506, the position (e.g., default and current) for the couch 108, or other information pertaining to operation of the RT system 100. In some embodiments, the RT system components 2040 may provide their respective information for storage in the LUT 2034 during the calibration phase and/or during real-time operation of the RT system 100.

The information sources 2030 may include other information from other sources 2042 additionally or alternatively to the information/sources previously described above. Such information from these outer sources 2042 may be used by the controller(s) 2010 to perform the positional adjustment and/or other operations of the RT system 100.

The position adjustment components 2020 may include one or more of: the magnet(s) 902 and related components for adjusting the position of the source 502, the components of the MLC 1102 for adjusting the field center 506, the couch positioning assembly 101 for adjusting the position of the couch 108, etc. The position adjustment components 2020 are responsive to and controllable by the controller(s) 2010 to perform the various positional adjustment operations (and related operations) described herein.

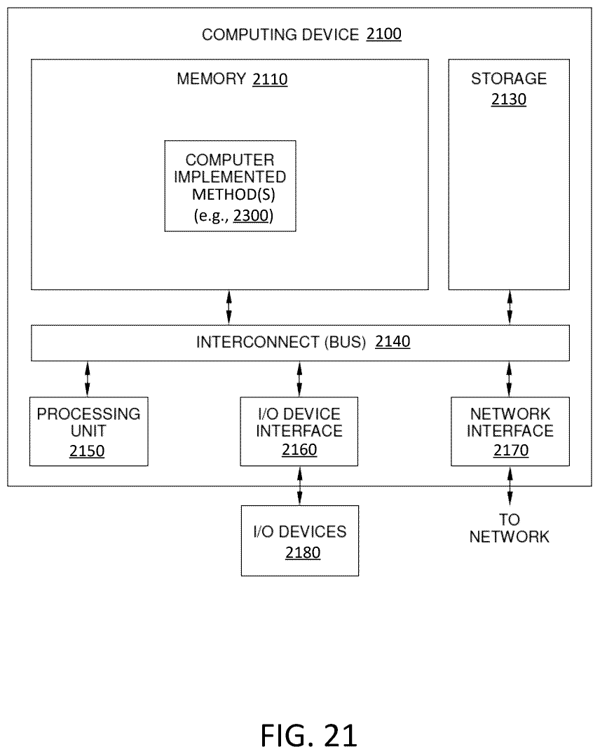

FIG. 21 is a block diagram of a computing device 2100 configured to perform various embodiments of the present disclosure. The computing device 2100 may be a desktop computer, a laptop computer, a smart phone, or any other type of computing device suitable for practicing one or more embodiments of the present disclosure. In operation, the computing device 2100 is configured to execute instructions associated with positional adjustment and/or other methods or operations, as described herein. It is noted that the computing device 2100 described herein is illustrative and that any other technically feasible configurations fall within the scope of the present disclosure.

As shown, the computing device 2100 includes, without limitation, an interconnect (bus) 2140 that connects a processing unit 2150, an input/output (I/O) device interface 2160 coupled to input/output (I/O) devices 2180, memory 2110, a storage 2130, and a network interface 2170. The processing unit 2150 may be any suitable processor implemented as a central processing unit (CPU), a graphics processing unit (GPU), an application-specific integrated circuit (ASIC), a field programmable gate array (FPGA), any other type of processing unit, or a combination of different processing units, such as a CPU configured to operate in conjunction with a GPU or digital signal processor (DSP). In general, the processing unit 2150 may be any technically feasible hardware unit capable of processing data and/or executing software applications, including the positional adjustment methods/operations described herein. In some embodiments, the processing unit 2150 may be used to implement one or more of the controllers 2010.

The I/O devices 2180 may include devices capable of providing input, such as a keyboard, a mouse, a touch-sensitive screen, and so forth, as well as devices capable of providing output, such as a display device and the like. Additionally, the I/O devices 2180 may include devices capable of both receiving input and providing output, such as a touchscreen, a universal serial bus (USB) port, and so forth. The I/O devices 2180 may be configured to receive various types of input from an end-user of the computing device 2100, and to also provide various types of output to the end-user of the computing device 2100, such as displayed digital images or digital videos. In some embodiments, one or more of the I/O devices 2180 are configured to couple the computing device 2100 to a network.

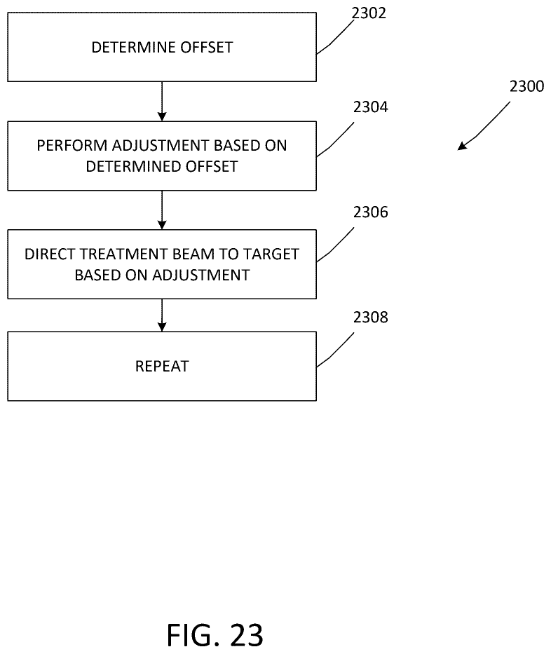

The memory 2110 may include a random access memory (RAM) module, a flash memory unit, or any other type of memory unit or combination thereof. The processing unit 2150, I/O device interface 2160, and network interface 2170 are configured to read data from and write data to the memory 2110. The memory 2110 includes various software programs that can be executed by processing unit 2150 and application data associated with said software programs, including the methods/operations described herein for reducing an isocenter size and for operating the RT system 100, including the positional adjustment methods/operations described herein. An example is described later below with respect to a method 2300 in FIG. 23.

The storage 2130 of various embodiments may store the LUT 2034 and/or other information from the information sources 2030. Also, some of this information/data may be stored in both the memory 2110 and the storage 2130.

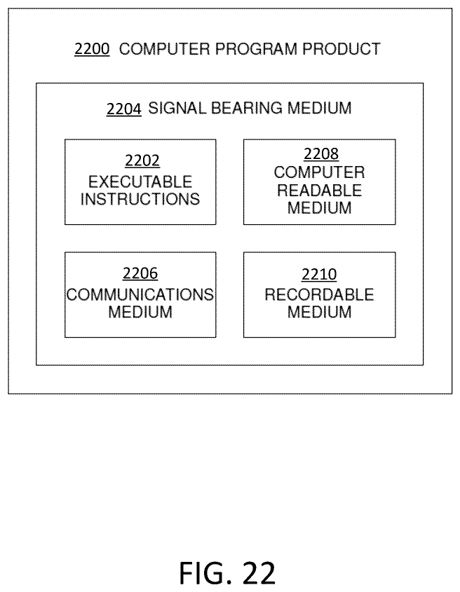

FIG. 22 is a block diagram of an illustrative embodiment of a computer program product 2200 for implementing a method, according to one or more embodiments of the present disclosure, such as the method and related operations described herein that pertain to positional adjustment for reducing isocenter size. The computer program product 2200 may be an article of manufacture that includes a signal bearing medium 2204. The signal bearing medium 2204 may include one or more sets of executable instructions 2202 that, when executed by, for example, a processor of a computing device, may provide at least the functionality described throughout this disclosure.

In some implementations, the signal bearing medium 2204 may encompass a tangible non-transitory computer readable medium 2208, such as, but not limited to, a hard disk drive, a compact disc (CD), a digital video disk (DVD), a digital tape, memory, etc. In some implementations, the signal bearing medium 2204 may encompass a recordable medium 2210, such as, but not limited to, memory, read/write (R/W) CDs, R/W DVDs, etc. In some implementations, the signal bearing medium 2204 may encompass a communications medium 2206, such as, but not limited to, a digital and/or an analog communication medium (e.g., a fiber optic cable, a waveguide, a wired communications link, a wireless communication link, etc.). The computer program product 2200 may be recorded on non-transitory computer readable medium 2208 or another similar recordable medium 2210.

FIG. 23 is a flowchart of an example method 2300 to reduce isocenter size (e.g., the isocenter size 602) for the RT system 100 and to operate the RT system 100, according to various embodiments. The example method 2300 may include one or more operations, functions, or actions illustrated by one or more blocks, such as blocks 2302 to 2308. The various blocks of the method 2300 and/or of any other process(es) described herein may be combined into fewer blocks, divided into additional blocks, supplemented with further blocks, and/or eliminated based upon the desired implementation. In one embodiment, the operations of the method 2300 and/or of any other process(es) described herein may be performed in a pipelined sequential manner. In other embodiments, some operations may be performed out-of-order, in parallel, etc.

According to one embodiment, at least some of the operations depicted in the method 2300 may be performed by the controller 2010 in cooperation with the other components of the RT system 100. The controller 2010 of various embodiments may instruct/cause other components in the RT system 100 to perform at least some of the operations depicted in the method 2300.

Starting at a block 2302 (“DETERMINE OFFSET”), the controller 2010 may determine, for each gantry angle, an offset of the central beam axis 305 of the treatment beam 230 relative to the isocenter 203, which may be an ideal isocenter in a situation where there is no downward bending of the gantry head of the C-arm gantry 110. However and as previously shown and described above, gravity may cause the gantry head to bend downwards, thereby causing an offset (e.g., a deviation or misalignment) of the central beam axis 305 relative to the isocenter 203, for each gantry angle.

The offset at the block 2302 may be determined using some of the example techniques described above (e.g., by using an image camera or other instrumentation), and then information pertaining to the determined offset may be stored in the LUT 2034 for each gantry angle. The stored information may include, for example, positional adjustment information for the source 502, the field center 506, or the couch 108 for each gantry angle, the coordinates of where the offset central beam axis 305 impinges in/on the radiation area 103, etc.).

In some embodiments, the offset determination performed at the block 2302 may be performed during a calibration phase of the RT system 100 and may be updated over time in subsequent calibration phases. In other embodiments, the information for the LUT 2034 (including the offset information) may be determined in a more dynamic manner at the block 2302, such as in real-time during operation of the RT system 100 while preparing to execute or currently executing a treatment plan.

The block 2302 may be followed by a block 2304 (“PERFORM ADJUSTMENT BASED ON DETERMINED OFFSET”) wherein the controller 2010 performs certain adjustment operations based on or in response to the determined offset. For example, the controller 2010 may reduce the isocenter size (e.g., compensate for the offset) by changing a position of the source 502, by changing a position of the field center 506, or by changing a position of the couch 108, in the manner described previously above with respect to FIGS. 7, 8, 9, 10, 11, 12, and 13. Also in some embodiments at the block 2302, the controller 2010 may perform an adjustment to adapt the isocenter size and/or location, by changing the position of the couch 108 in view of weights assigned to central beam axes 305 and/or other criteria set forth in a treatment plan, wherein different patients may have different treatment plans that are not based on a global ideal isocenter—such embodiments are described previously with respect to the examples of FIGS. 14, 15, 16, 17, 18, and 19.

In some embodiments, the adjustment performed at the block 2304 is a discrete/separate adjustment. For example, the position of the source 502 may be changed, while the position of the field center 506 is unchanged. In other embodiments, a combination of different types of adjustments may be performed. For example, a central beam axis 305 may be tuned to an isocenter by adjusting positions of both the source 502 and the field center 506, rather than by adjusting the position of just one of them.

The block 2304 may be followed by a block 2306 (“DIRECT TREATMENT BEAM TO TARGET BASED ON ADJUSTMENT”) in which the treatment beam 230 is directed towards a target based on the type of adjustment that has been performed. For example, if the target has been placed on the isocenter 203, then the central beam axis 305 of the treatment beam 230 is directed to and impinges on (e.g., coincides with) the isocenter, due to the position of the source 502 or the field center 506 having been changed. As another example, the position of the couch 108 can be changed so as to place the target in coincidence with where the central beam axis 305 is offset from the isocenter 203. As still another example, the position of the couch 108 can be changed based on assigned weights (specified in a treatment plan) that dictate where to place the target relative of one or more central beam axes 305 that are offset from the ideal or common isocenter 203.

The block 2306 may be followed by a block 2308 (“REPEAT”), in which one or more of the operations previously described above may be repeated. For example, determination of the offset (such as in a calibration phase) may be performed again to determine if there are changes in the offset and/or to otherwise ensure the continued accuracy of the RT system 100. Also at the block 2308, directing the treatment beam 230 may be repeated for other gantry angles, for the same or other patient(s) and/or treatment plans.

In sum, embodiments described herein provide techniques to reduce isocenter size, thereby reducing the maximum delivery error. The target (e.g., a tumor) may be placed at a location of an ideal isocenter, and the central beam axes 305 of the treatment beam 230 can be tuned to that isocenter. The isocenter size may also be reduced by placing the target (by changing the position of the couch 108) in coincidence with an impingement location where the central beam axis 305 is offset from the ideal isocenter. Furthermore, the placement of the target (by the positioning of the couch 108) may be based on weights of the central beam axes 305 as specified in a treatment plan.

The descriptions of the various embodiments have been presented for purposes of illustration, but are not intended to be exhaustive or limited to the embodiments disclosed. Many modifications and variations are possible without departing from the scope and spirit of the described embodiments.

Aspects of the present embodiments may be embodied as a system, method, or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

While various aspects and embodiments have been disclosed herein, other aspects and embodiments are possible. The various aspects and embodiments disclosed herein are for purposes of illustration and are not intended to be limiting, with the true scope and spirit being indicated by the following claims.

1. A radiation therapy (RT) system, comprising:
a gantry having a linear accelerator, wherein the gantry is rotationally movable to position the gantry at a plurality of gantry angles, wherein at any of the gantry angles, the linear accelerator is configured to generate a treatment beam having a central beam axis, and wherein each treatment beam is assigned a respective weight in accordance with at least one treatment plan;
a couch that provides a target where the treatment beam is directed; and
a controller operatively coupled to the couch and configured to position the target relative to the treatment beam based at least in part on the weight assigned by the at least one treatment plan, wherein the controller is further configured to maintain the target in a static position relative to the treatment beam during treatment.

2. The RT system of claim 1, wherein respective treatment plans for a plurality of patients specify different weighting strategies for the treatment beams, wherein the controller is configured to position the target based on weights assigned by the weighting strategies, and wherein the position of the target based on the weights provides an isocenter for each patient that is different from the isocenter which is common to all of the patients.

3. The RT system of claim 1, wherein the at least one treatment plan includes a weighting strategy that specifies one or more of:
infinite weight assigned to all central beam axes,
uniform weight assigned to all central beam axes,
a user-defined weight assigned to each central beam axis,
a percentage of monitor unit (MU) delivered along each central beam axes, or
a robustness metric that defines how robust the treatment beam is to geometric uncertainties.

4. The RT system of claim 1, wherein:
the gantry includes a gantry head that bends downward due to gravity, and
for each gantry angle of the plurality of gantry angles, the downward bend of the gantry head offsets the central beam axis relative to an isocenter that is common to all of the treatment beams.

5. The RT system of claim 4, wherein:
the offset of the central beam axis relative to the isocenter corresponds to a cosine function of deviation versus gantry angle, and
the at least one treatment plan specifies which gantry angles and deviation of the cosine function to use by the controller as a basis to position the target.

6. The RT system of claim 1, wherein based at least in part on the weight assigned by the at least one treatment plan, the controller is configured to position the target relative to the treatment beam so that the target is closer to the central beam axis of the treatment beam, but not coincident with the central beam axis, and further away from the central beam axes of other treatment beams.

7. The RT system of claim 1, wherein based at least in part on the weight assigned by the at least one treatment plan, the controller is configured to position the target relative to the treatment beam so that the target is coincident with the central beam axis of the treatment beam and further away from the central beam axes of other treatment beams.

8. A radiation therapy (RT) system, comprising:
a gantry having a linear accelerator, wherein the gantry is rotationally movable to position the gantry at a plurality of gantry angles, wherein at any of the gantry angles, the gantry is configured to direct a treatment beam generated by the linear accelerator towards a target, and wherein the treatment beam at each of the plurality of gantry angles is associated with a weighting strategy specified in at least one treatment plan;
a source of the treatment beam at the linear accelerator;
a collimator at the gantry and having a field center, wherein a central beam axis of the treatment beam is defined by the source and the field center;
a couch that provides the target where the treatment beam is directed; and
a controller operatively coupled to the couch, wherein the controller is configured to position the couch to align the target with the central beam axis, wherein the alignment of the target with the central beam axis is based at least in part on the weighting strategy, and wherein the controller is further configured to statically maintain the alignment of the target with the central beam axis during treatment.

9. The RT system of claim 8, wherein respective treatment plans for a plurality of patients specify different weighting strategies for the treatment beam at each of the plurality of gantry angles, and wherein the alignment of the target based at least in part on the weighting strategy provides a different isocenter for each patient that is different from the isocenter which is common to all of the patients.

10. The RT system of claim 8, wherein positions of either or both the source and the field center are unchanged during the treatment.

11. The RT system of claim 8, wherein the weighting strategy specifies one or more of:
infinite weight assigned to all central beam axes,
uniform weight assigned to all central beam axes,
a user-defined weight assigned to each central beam axis,
a percentage of monitor unit (MU) delivered along each central beam axes, or
a robustness metric that defines how robust the treatment beam is to geometric uncertainties.

12. The RT system of claim 8, wherein offset location of the central beam axis relative to an isocenter corresponds to a cosine function of deviation versus gantry angle, and wherein the at least one treatment plan specifies which gantry angles and deviation of the cosine function to use by the controller as a basis to align the target with the central beam axis.

13. The RT system of claim 8, wherein:
the gantry includes a gantry head that bends downward due to gravity, and
for each gantry angle of the plurality of gantry angles, the downward bend of the gantry head misaligns the central beam axis from an isocenter common to all central beam axes to an offset location.

14. The RT system of claim 8, further comprising a lookup table that indicates, for each gantry angle, an offset location of the central beam axis relative to an isocenter common to all central beam axis, wherein the controller is configured to access the lookup table to determine the offset location for alignment of the target to the central beam axis.

15. A computer-implemented method to adapt a location and size of an isocenter in a radiation therapy (RT) system, the method comprising:
determining a weight assigned by at least one treatment plan to a central beam axis of a treatment beam that deviates relative to the isocenter, wherein the RT system includes a gantry having a linear accelerator configured to generate the treatment beam, wherein the gantry is rotationally movable to position the gantry at a plurality of gantry angles, and wherein at any particular gantry angle of the gantry angles, the gantry is configured to provide the treatment beam;
positioning a target of the treatment beam relative to the central beam axis based at least in part on the weight assigned by the at least one treatment plan; and
statically maintaining the positioning of the target relative to the central beam axis during treatment.

16. The method of claim 15, wherein positioning the target comprises moving the target closer to the central beam axis based at least in part on the weight assigned to the central beam axis being higher than weights assigned to other central beam axes.

17. The method of claim 15, wherein positioning the target comprises moving the target further away from the central beam axis based at least in part on the weight assigned to the central beam axis being lower than weights assigned to other central beam axes.

18. The method of claim 15, wherein respective treatment plans for a plurality of patients specify different weighting strategies for the treatment beam at each of the plurality of gantry angles, and wherein the weighting strategies provide a different isocenter for each patient that is different in location and size from the isocenter which is common to all of the patients.

19. The method of claim 15, further comprising:
storing, in a lookup table, information that indicates the deviation of the central beam axis at each gantry angle; and
accessing the lookup table to determine the positioning of the target relative to the deviation of the central beam axis.

20. The method of claim 15, wherein:
the gantry includes a gantry head that bends downward due to gravity, and for each gantry angle of the plurality of gantry angles, the downward bend of the gantry head deviates the central beam axis from the isocenter.
